(19) United States
(12) Patent Application Publication
(10) Pub. No.: US 2006/0035941 A1
(43) Pub. Date: Feb. 16, 2006

(54) Calcium salts of indole derived statins

(76) Inventors: Guang-Pei Chen, Livingston, NJ (US); Prasad Koteswara Kapa, Parsippany, NJ (US); Paul Allen Sutton, Parsippany, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/517,874

(22) PCT Filed: Jun. 12, 2003
(86) PCT No.: PCT/EP03/06195

§ 371 Date: Dec. 13, 2004

Related U.S. Application Data
(60) Provisional application No. 60/388,318, filed on Jun. 13, 2002.

(51) Int. Cl. A61K 31/404; C07D 209/04

(52) U.S. Cl. 514/359; 514/415; 548/510

(57) ABSTRACT

The present invention provides calcium salts of indole derived statins of the formula (IA) wherein R₁, is alkyl, cycloalkyl or aralkyl; R₂, R₃ and R₄ are independently hydrogen, halogen or alkyl; R₅ and R₆ are independently hydrogen, halogen, alkyl, cycloalkyl, aralkyl, alkoxy or aralkoxy; and the hydroxyl group at the 3position is in the R-configuration and at the 5position in the S-configuration; or an enantiomer thereof; or a hydrate thereof; as obtainable by the methods of the present invention. More specifically, the invention provides a calcium salt of formula (IA) wherein R₁, is isopropyl, R₂ is fluorine and R₃, R₄, R₅ and R₆ are hydrogen, designated herein as Fluvastatin calcium, in a highly crystalline form. Furthermore, the present invention is directed to methods for the preparation of the crystalline Fluvastatin calcium, and to pharmaceutical compositions comprising the crystalline form.

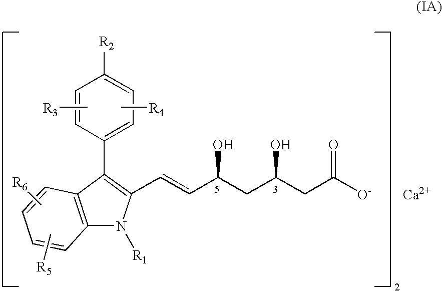

Fluvastatin, (3R*,5S*)-(E)-7-[3-(4-fluorophenyl)-1-(1-methyl-ethyl)-1H-indol-2-yl]-3,5-dihydroxy-6-heptenoic acid of the formula 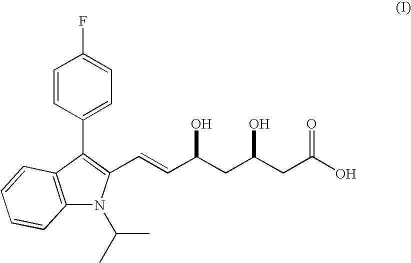
or pharmaceutically acceptable salts thereof, are inhibitors of 3-hydroxy-3-methyl-glutaryl coenzyme A (HMG-CoA) reductase. Thus, Fluvastatin, in any of its forms, may be employed for lowering of blood cholesterol levels and, therefore, for the treatment of conditions such as hypercholesteremia, hyperlipoproteinemia, dyslipidemia and atherosclerosis.

Both the racemate as well as the single enantiomers of Fluvastatin, and the sodium salts thereof, also known as Fluvastatin sodium, are disclosed in U.S. Pat. No. 5,354,772. The publication by Tempkin et. al. (Tetrahedron 1997, vol. 53, 10659-10670) describes an asymmetric synthesis of the biologically most potent (3R, 5S) isomer. U.S. Pat. No. 4,739,073 discloses isolation of Fluvastatin as its sodium salt by lyophilization. However, as disclosed in EP 547 000, Fluvastatin sodium is an amorphous solid and extremely susceptible to degradation at a pH about 8 or below. The suggested solution is to provide pharmaceutical compositions comprising the drug substance and an alkaline medium which is capable of sustaining the aqueous solution or dispersion of the pharmaceutical composition at a pH of at least 8. In addition to the pH sensitivity, heat and light sensitivity, as well as hygroscopicity of Fluvastatin sodium impose particular requirements on the manufacture and storage of pharmaceutical compositions comprising Fluvastatin sodium.

It is known in the art that higher crystallinity and lower hygroscopicity will lead to improved chemical stability and longer shelf life of chemical compounds. Consequently, there is a need for new forms of Fluvastatin having improved chemical stability, making possible the preparation of pharmaceutical formulations of Fluvastatin with less need for stabilizing agents and with prolonged shelf life, and with the possibility of being provided in less sophisticated packages.

In one aspect, the present invention provides calcium salts of the formula 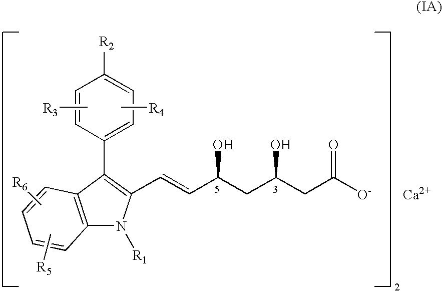
wherein R₁ is alkyl, cycloalkyl or aralkyl; R₂, R₃ and R₄ are independently hydrogen, halogen or alkyl; R₅ and R₆ are independently hydrogen, halogen, alkyl, cycloalkyl, aralkyl, alkoxy or aralkoxy; and the hydroxyl group at the 3-position is in the R-configuration and at the 5-position in the S-configuration; or an enantiomer thereof; or a hydrate thereof.

In a preferred embodiment of the present invention the calcium salt is a compound of formula IA wherein R₁ is isopropyl; R₂ is fluorine and R₃, R₄, R₅ and R₆ are hydrogen; designated herein as Fluvastatin calcium.

In another aspect, the present invention relates to a crystalline form of Fluvastatin, more specifically, a crystalline Fluvastatin calcium, and to methods for the preparation of such form of Fluvastatin.

In a further aspect, the present invention provides pharmaceutical compositions comprising crystals of Fluvastatin calcium as obtainable by the methods of the present invention, and pharmaceutically acceptable excipients, diluents or carriers thereof.

Other aspects of the present invention will become apparent to those skilled in the art from the following description, appended claims and accompanying drawings. It should be understood, however, that the following description, appended claims and specific examples, while indicating preferred embodiments of the invention, are given by way of illustration only. Various changes and modifications within the spirit and scope of the disclosed invention will become readily apparent to those skilled in the art from reading the following.

The present invention relates to calcium salts of indole derived statins, more specifically, to optically active calcium salts of Fluvastatin, designated herein as Fluvastatin calcium, and to methods for the preparation of such forms of Fluvastatin, and to pharmaceutical compositions comprising the crystalline forms of Fluvastatin.

Listed below are definitions of various terms used to describe the compounds of the present invention. These definitions apply to the terms as they are used throughout the specification unless they are otherwise limited in specific instances either individually or as part of a larger group.

The term “alkyl” refers to straight or branched chain hydrocarbon groups having 1 to 20 carbon atoms, preferably 1 to 7 carbon atoms. Exemplary unsubstituted alkyl groups include methyl, ethyl, propyl, isopropyl, n-butyl, t-butyl, isobutyl, pentyl, hexyl, isohexyl, heptyl, 4,4-dimethylpentyl, octyl and the like.

The term “lower alkyl” refers to those alkyl groups as described above having 1 to 7, preferably 1 to 4 carbon atoms.

The term “halogen” or “halo” refers to fluorine, chlorine, bromine and iodine.

The term “cycloalkyl” refers to optionally substituted monocyclic, bicyclic or tricyclic hydrocarbon groups of 3 to 12 carbon atoms, each of which may be substituted by one or more substituents such as alkyl, halo, oxo, hydroxy, alkoxy, alkylthio, nitro, cyano, trifluoromethyl and the like.

Exemplary monocyclic hydrocarbon groups include but are not limited to cyclopropyl, cyclobutyl, cyclopentyl, cyclopentenyl, cyclohexyl and cyclohexenyl and the like.

Exemplary bicyclic hydrocarbon groups include bornyl, indyl, hexahydroindyl, tetrahydronaphthyl, decahydronaphthyl, bicyclo[2.1.1]hexyl, bicyclo[2.2.1]heptyl, bicyclo[2.2.1]heptenyl, 6,6-dimethylbicyclo[3.1.1]heptyl, 2,6,6-trimethylbicyclo[3.1.1]heptyl, bicyclo[2.2.2]octyl and the like.

Exemplary tricyclic hydrocarbon groups include adamantyl and the like.

The term “alkoxy” refers to alkyl-O—.

The term “alkylthio” refers to alkyl-S—.

The term “aralkyl” refers to an aryl group bonded directly through an alkyl group, such as benzyl and phenethyl.

The term “aralkoxy” refers to an aryl group bonded directly through an alkoxy group.

The term “aryl” refers to monocyclic or bicyclic aromatic hydrocarbon groups having 6 to 12 carbon atoms in the ring portion, such as phenyl, naphthyl, tetrahydronaphthyl, biphenyl and diphenyl groups, each of which may optionally be substituted by one to four substituents such as alkyl, halo, hydroxy, alkoxy, thiol, alkylthio, nitro, cyano and the like.

In one aspect, the present invention provides calcium salts of the formula 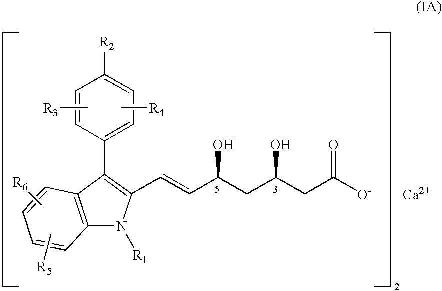
wherein R₁ is alkyl, cycloalkyl or aralkyl; R₂, R₃ and R₄ are independently hydrogen, halogen or alkyl; R₅ and R₆ are independently hydrogen, halogen, alkyl, cycloalkyl, aralkyl, alkoxy or aralkoxy; and the hydroxyl group at the 3-position is in the R-configuration and at the 5-position in the S-configuration; or an enantiomer thereof; or a hydrate thereof; which are prepared by first hydrolyzing a compound of the formula 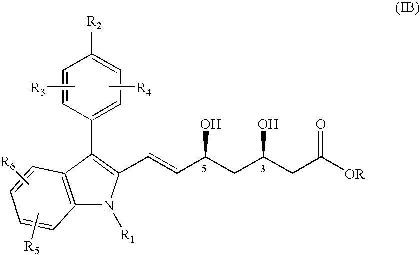
wherein R₁, R₂, R₃, R₄, R₅ and R₆ have meanings as defined for formula IA; R represents lower alkyl; and the hydroxyl group at the 3-position is in the R-configuration and at the 5-position in the S-configuration; or an enantiomer thereof; in the presence of an aqueous base, preferably an alkali metal hydroxide, to afford alkali metal salts of the formula 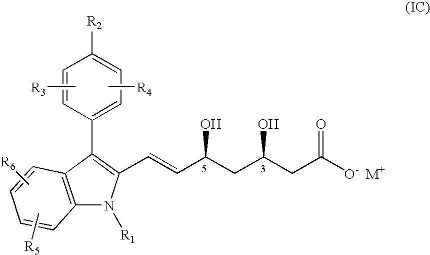
wherein M represents sodium, lithium or potassium, preferably M is sodium.

Alkali metal salts of formula IC wherein R₁, R₂, R₃, R₄, R₅, R₆ and M have meanings as defined herein above; and the hydroxyl group at the 3-position is in the R-configuration and at the 5-position in the S-configuration; or an enantiomer thereof; or a hydrate thereof; are then treated with a suitable calcium compound to afford the corresponding calcium salt of formula IA. Suitable calcium compounds include, but are not limited to, calcium chloride, calcium oxide and calcium hydroxide. Preferably, the calcium compound is calcium chloride.

In a preferred embodiment of the present invention said calcium salt is a compound of formula IA wherein R₁ is isopropyl; R₂ is fluorine; and R₃, R₄, R₅ and R₆ are hydrogen; designated herein as Fluvastatin calcium.

In a further aspect, the present invention provides a crystalline form of Fluvastatin, more specifically, a crystalline form of Fluvastatin calcium, and methods for the preparation of such form of Fluvastatin.

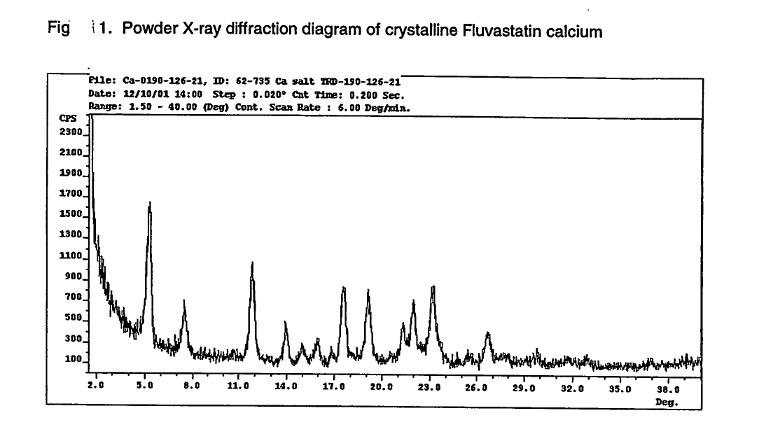

In particular, the present invention is directed to a crystalline Fluvastatin calcium which is characterized by the following physical properties: the powder X-ray diffraction diagram shows maxima at 2θ values of 5.3, 11.8, 13.9, 17.5, 19.1, 22.0 and 23.1 (FIG. 1); and the melting point has been found to be about 220° C.

The melting point has been measured by Differential Scanning Calorimetry (DSC) method using Seiko DSC 22C system, and the powder X-ray diffraction diagram has been recorded between 1.5° and 40° (2 theta, i.e., 2θ) with CuK∝ radiation using a Scintag XDS diffraction system.

A discussion of the theory of powder X-ray diffraction patterns can be found in “X-ray diffraction procedures” by Klug and Alexander, J. Wiley, New York (1974), and as pointed out, e.g., by Li et. al. in J. Pharm. Sci., pages 337-346 (1999), enantiomers have same solid state properties, like melting points and X-ray data.

The crystalline Fluvastatin calcium defined herein above may be prepared as described generally herein for calcium salts of formula IA, i.e., by first hydrolyzing a compound of the formula 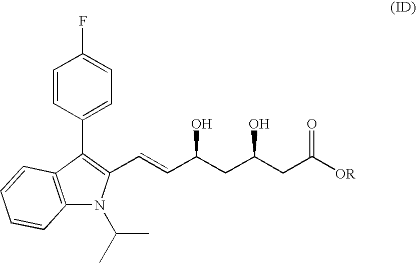
wherein R represents lower alkyl, preferably methyl, ethyl or t-butyl; and the hydroxyl group at the 3-position is in the R-configuration and at the 5-position in the S-configuration; or an enantiomer thereof; in the presence of an aqueous base, preferably an alkali metal hydroxide, to afford alkali metal salts of the formula 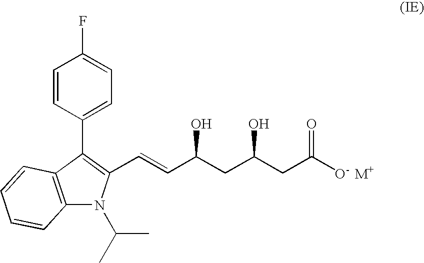
wherein M represents sodium, lithium or potassium, preferably sodium.

The hydrolysis step above may be carried out in the presence of an organic solvent such as a lower alcohol, preferably ethanol, and the aqueous base used to accomplish the hydrolysis is preferably selected from the group consisting of potassium hydroxide, lithium hydroxide and sodium hydroxide. More preferably, the base is sodium hydroxide. The hydrolysis is preferably conducted at a temperature ranging from about 0° C. to about 45° C., preferably from about 20° C. to about 35° C.

Alkali metal salts of formula IE may be isolated by lyophilization as described for Fluvastatin sodium in U.S. Pat. No. 4,739,073, and then converted to the crystalline Fluvastatin calcium defined herein above, or preferably, the alkali metal salts of formula IE may be converted to the crystalline Fluvastatin calcium without isolation as described herein in the illustrative Examples.

Accordingly, the crystalline Fluvastatin calcium may be obtained by reacting an aqueous solution of an alkali metal salt of formula IE, preferably a sodium salt of formula IE, with an aqueous solution of a suitable calcium compound at an ambient temperature, preferably at room temperature. Suitable calcium compounds include, but are not limited to, calcium chloride, calcium oxide and calcium hydroxide. Preferably, the calcium compound is calcium chloride.

The resulting precipitated crystals of Fluvastatin calcium, or a hydrate thereof, may be isolated using conventional methods such as vacuum filtration or centrifugation, preferably vacuum filtration. The crystals may be washed, preferably with water, and then dried under atmospheric or reduced pressure, preferably under reduced pressure ranging from about 20 to about 0.1 mmHg, at a temperature ranging from room temperature to about 50° C. for a period ranging from about 24 to about 72 h.

The processes described herein above may be conducted under inert atmosphere, preferably under nitrogen atmosphere.

The above disclosed methods for the preparation of Fluvastatin calcium give Fluvastatin in a stable crystalline form which is preferable to Fluvastatin sodium produced by lyophilization as an amorphous solid.

In a further aspect, the present invention provides pharmaceutical compositions comprising crystals of Fluvastatin calcium, e.g. as obtainable by the above methods, and pharmaceutically acceptable excipients, diluents or carriers thereof, especially for the prevention and/or treatment e.g. of hypercholesterolemia, hyperlipoproteinemia, dyslipidemia and atherosclerosis in mammals.

The present invention also relates to the use of Fluvastatin calcium in the manufacture of a pharmaceutical composition comprising mixing an effective amount of crystals of Fluvastatin calcium as obtainable by the above methods, and pharmaceutically acceptable excipients, diluents or carriers thereof, especially for the prevention and/or treatment e.g. of hyper-cholesterolemia, hyperlipoproteinemia, dyslipidemia and atherosclerosis in mammals.

Fluvastatin, in any of its forms, may be employed therapeutically for lowering of blood cholesterol levels, and therefore, as a hypercholesterolemic, hyperlipoproteinemic, dyslipidemic and antiatherosclerotic agent. Accordingly, the present invention provides a method for the prevention and/or treatment e.g. of hypercholesterolemia, hyperlipoproteinemia, dyslipidemia and atherosclerosis in mammals, which method comprises administering a therapeutically effective amount of crystals of Fluvastatin calcium as obtainable by the methods of the present invention.

The invention further relates to a combination, such as a pharmaceutical composition, comprising a salt of formula (I A) and at least one therapeutic agent selected from the group consisting of
(i) an AT₁-receptor antagonist or a pharmaceutically acceptable salt thereof,
(ii) a renin inhibitor or a pharmaceutically acceptable salt thereof,
(iii) an angiotensin converting enzyme (ACE) inhibitor or a pharmaceutically acceptable salt thereof,
(iv) an Calcium channel blocker or a pharmaceutically acceptable salt thereof,
(v) an aldosterone synthase inhibitor or a pharmaceutically acceptable salt thereof,
(vi) an aldosterone antagonist or a pharmaceutically acceptable salt thereof,
(vii) an dual angiotensin converting enzyme/neutral endopetidase (ACE/NEP) inhibitor or a pharmaceutically acceptable salt thereof,
(viii) an endothelin antagonist or a pharmaceutically acceptable salt thereof, and
(ix) a diuretic or a pharmaceutically acceptable salt thereof.

The term “at least one therapeutic agent” shall mean that in addition to the compound of formula (I) one or more, for example two, furthermore three, active ingredients as specified according to the present invention can be combined.

AT₁-receptor antagonists (also called angiotensin II receptor antagonists) are understood to be those active ingredients that bind to the AT₁-receptor subtype of angiotensin II receptor but do not result in activation of the receptor. As a consequence of the inhibition of the AT, receptor, these antagonists can, for example, be employed as antihypertensives or for treating congestive heart failure.

The class of AT₁ receptor antagonists comprises compounds having differing structural features, essentially preferred are the non-peptidic ones. For example, mention may be made of the compounds that are selected from the group consisting of valsartan (cf. EP 443983), losartan (cf. EP253310), candesartan (cf. 459136), eprosartan (cf. EP 403159), irbesartan (cf. EP454511), olmesartan (cf. EP 503785), tasosartan (cf. EP539086), telmisartan (cf. EP 522314), the compound with the designation E-1477 of the following formula 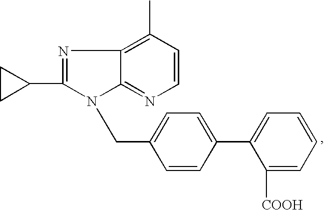
the compound with the designation SC-52458 of the following formula 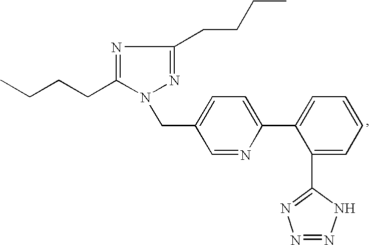
and the compound with the designation the compound ZD-8731 of the following formula 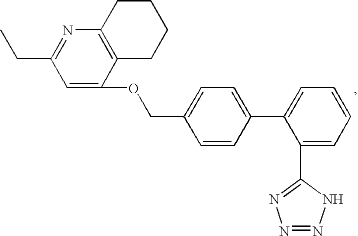
or, in each case, a pharmaceutically acceptable salt thereof.

Preferred AT₁-receptor antagonist are those agents that have been marketed, most preferred is valsartan or a pharmaceutically acceptable salt thereof.

A preferred renin is aliskiren, chemically defined as 2(S),4(S),5(S),7(S)-N-(3-amino-2,2-dimethyl-3-oxopropyl)-2,7-di(1-methylethyl)-4-hydroxy-5-amino-[4-methoxy-3-(3-methoxy-propoxy)phenyl]-octanamide, is specifically disclosed in EP 678503 A. Especially preferred is the hemi-fumarate salt thereof.

The interruption of the enzymatic degradation of angiotensin I to angiotensin II with so-called ACE-inhibitors (also called angiotensin converting enzyme inhibitors) is a successful variant for the regulation of blood pressure and thus also makes available a therapeutic method for the treatment of congestive heart failure.

The class of ACE inhibitors comprises compounds having differing structural features. For example, mention may be made of the compounds which are selected from the group consisting alacepril, benazepril, benazeprilat, captopril, ceronapril, cilazapril, delapril, enalapril, enaprilat, fosinopril, imidapril, lisinopril, moveltopril, perindopril, quinapril, ramipril, spirapril, temocapril, and trandolapril, or, in each case, a pharmaceutically acceptable salt thereof.

Preferred ACE inhibitors are those agents that have been marketed, most preferred are benazepril and enalapril.

The class of CCBs essentially comprises dihydropyridines (DHPs) and non-DHPs such as diltiazem-type and verapamil-type CCBs.

A CCB useful in said combination is preferably a DHP representative selected from the group consisting of amlodipine, felodipine, ryosidine, isradipine, lacidipine, nicardipine, nifedipine, niguldipine, niludipine, nimodipine, nisoldipine, nitrendipine, and nivaldipine, and is preferably a non-DHP representative selected from the group consisting of flunarizine, prenylamine, diltiazem, fendiline, gallopamil, mibefradil, anipamil, tiapamil and verapamil, and in each case, a pharmaceutically acceptable salt thereof. All these CCBs are therapeutically used, e.g. as anti-hypertensive, anti-angina pectoris or anti-arrhythmic drugs.

Preferred CCBs comprise amlodipine, diltiazem, isradipine, nicardipine, nifedipine, nimodipine, nisoldipine, nitrendipine, and verapamil, or, e.g. dependent on the specific CCB, a pharmaceutically acceptable salt thereof. Especially preferred as DHP is amlodipine or a pharmaceutically acceptable salt, especially the besylate, thereof. An especially preferred representative of non-DHPs is verapamil or a pharmaceutically acceptable salt, especially the hydrochloride, thereof.

Aldosterone synthase inhibitor is an enzyme that converts corticosterone to aldosterone by hydroxylating cortocosterone to form 18-OH-corticosterone and 18-OH-corticosterone to aldosterone. The class of aldosterone synthase inhibitors is known to be applied for the treatment of hypertension and primary aldosteronism comprises both steroidal and non-steroidal aldosterone synthase inhibitors, the later being most preferred.

Preference is given to commercially available aldosterone synthase inhibitors or those aldosterone synthase inhibitors that have been approved by the health authorities.

The class of aldosterone synthase inhibitors comprises compounds having differing structural features. For example, mention may be made of the compounds which are selected from the group consisting of the non-steroidal aromatase inhibitors anastrozole, fadrozole (including the (+)-enantiomer thereof), as well as the steroidal aromatase inhibitor exemestane, or, in each case where applicable, a pharmaceutically acceptable salt thereof.

The most preferred non-steroidal aldosterone synthase inhibitor is the (+)-enantiomer of fadrozole (U.S. Pat. Nos. 4,617,307 and 4,889,861) of formula 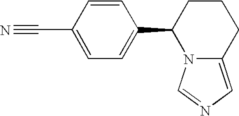
or a pharmaceutically acceptable salt thereof.

A preferred steroidal aldosterone antagonist is eplerenone (cf. EP 122232 A) of the formula 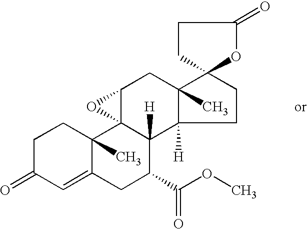
spironolactone.

Compounds having an inhibitory effects on both angiotensin converting enzyme and neutral endopetidase, so-called dual ACEINEP inhibitors, can be used for the treatment of cardiovascular pathologies.

A preferred dual angiotensin converting enzyme/neutral endopetidase (ACE/NEP) inhibitor is, for example, omapatrilate (cf. EP 629627), fasidotril or fasidotrilate, or Z 13752A (cf. WO 97/24342) or, if appropriable, a pharmaceutically acceptable salt thereof.

Endothelin (ET) is a highly potent vasoconstrictor peptided synthesized and released by the vascular endotleium. Endothelin exists in three isoforms (ET-1, ET-2 and ET-3). (ET shall meand any or all othe isoforms of ET). Elevated levels of ET have been reported in plasma fomr patients with e.g. essential hypertension. Endothelin receptor antagonist can be used to inhibit the vasoconstrictive effects induced by ET.

A preferred endothelin antagonist is, for example, bosentan (cf. EP 526708 A), enrasentan (cf. WO 94/25013), atrasentan (cf. WO 96/06095), especially atrasentan hydrochloride, darusentan (cf. EP 785926 A), BMS 193884 (cf. EP 702012 A), sitaxentan (cf. U.S. Pat. No. 5,594,021), especially sitaxsentan sodium, YM 598 (cf. EP 882719 A), S 0139 (cf. WO 97/27314), J 104132 (cf. EP 714897 A or WO 97/37665), furthermore, tezosentan (cf. WO 96/19459), or in each case, a pharmaceutically acceptable salt thereof.

A diuretic is, for example, a thiazide derivative selected from the group consisting of chlorothiazide, hydrochlorothiazide, methylclothiazide, and chlorothalidon. The most preferred is hydrochlorothiazide.

The structure of the active agents identified by generic or tradenames may be taken from the actual edition of the standard compendium “The Merck Index” or from databases, e.g. LifeCycle Patents International (e.g. IMS World Publications). The corresponding content thereof is hereby incorporated by reference. Any person skilled in the art is fully enabled to identify the active agents and, based on these references, likewise enabled to manufacture and test the pharmaceutical indications and properties in standard test models, both in vitro and in vivo.

The present invention is further described by the following examples. The examples are intended to illustrate the invention and are not to be construed as being limitations thereon.

If not mentioned otherwise, all evaporations are performed under reduced pressure, preferably between about 10 and 100 mmHg. The structure of final products, intermediates and starting materials is confirmed by standard analytical methods, e.g., microanalysis, melting point (mp) and spectroscopic characteristics (e.g. MS, IR, NMR).

### EXAMPLE 1

### (E)-(3R,5S)-(+)-7-[3-(4-Fluoro-phenyl)-1-isopropyl-1H-indol-2-yl]-3,5-dihydroxy-hept-6-enoic acid calcium salt, Fluvastatin calcium, (+)-enantiomer

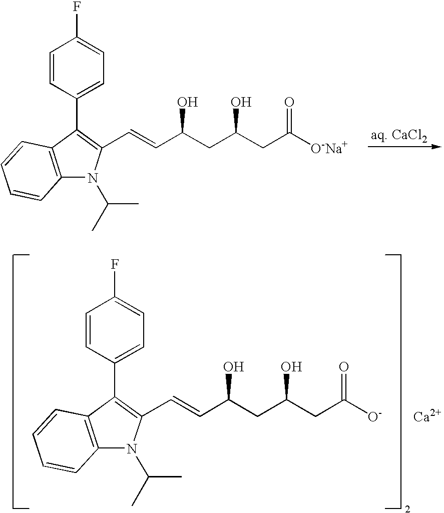

Under a nitrogen atmosphere, to a stirred solution of 15.0 g (0.0346 mol) of (E)-(3R,5S)-(+)-7-[3-(4-fluoro-phenyl)-1-isopropyl-1H-indol-2-yl]-3,5-dihydroxy-hept-6-enoic acid sodium salt in 300 mL of water is added a solution of 2.8 g (0.019 mol) of calcium chloride dihydrate in 20 mL of water at room temperature. A white precipitate starts to form immediately, and the reaction mixture gradually changes to a thick white slurry. After about 2 h, the precipitate is collected by vacuum filtration, washed twice with 50 mL of water and dried at 45° C. in a vacuum oven for about 72 h to afford 14.05 g of crystalline (E)-(3R,5S)-(+)-7-[3-(4-fluoro-phenyl)-1-isopropyl-1H-indol-2-yl]-3,5-dihydroxy-hept-6-enoic acid calcium salt: mp 220° C.; powder X-ray diffraction diagram as shown in FIG. 1.

### EXAMPLE 2

### (E)-(3R,5S)-(+)-7-[3-(4-Fluoro-phenyl)-1-isopropyl-1H-indol-2-yl]-3,5-dihydroxy-hept-6-enoic acid calcium salt, Fluvastatin calcium, (+)-enantiomer

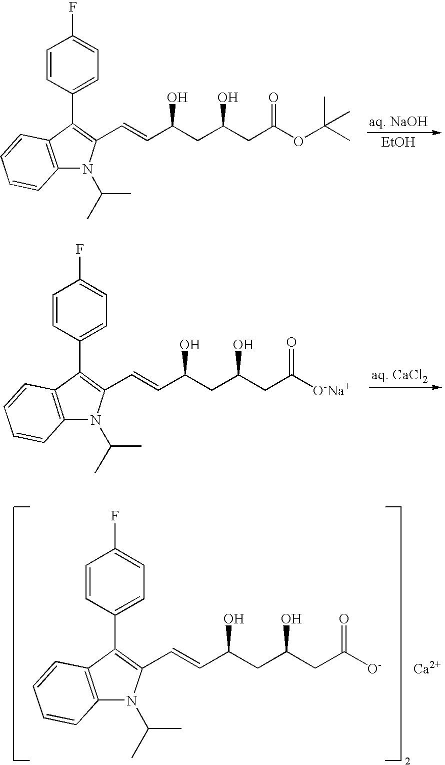

Under a nitrogen atmosphere, to a solution of 19.22 g (0.0412 mol) of (E)-(3R,5S)-(+)-7-[3-(4-fluoro-phenyl)-1-isopropyl-1H-indol-2-yl]-3,5-dihydroxy-hept-6-enoic acid 1,1-dimethylethyl ester in 95 mL of ethanol is added a solution of 1.58 g (0.0395 mol) of sodium hydroxide in 33 mL of water dropwise while maintaining the internal temperature in a range of 25±3° C. The reaction is then warmed to 33±2° C. and stirred for 1.5 h further. The reaction mixture is filtered and the filtration residue is washed with 20 mL of water. To the filtrate are added 120 mL of water and 220 mL of methyl-t-butyl ether, and the mixture is stirred for 5 min. The layers are separated, and the aqueous layer is washed with 75 mL of methyl-t-butyl ether. The aqueous solution is concentrated to a volume of about 100 mL under reduced pressure (water bath ˜50° C.), and 206 mL of water are added to form a yellow clear sodium salt solution. A solution of 3.21 g (0.0218 mol) of calcium chloride dihydrate in 40 mL of water is added dropwise while vigorously stirring. The solution immediately changes to a white slurry. Stirring is continued for 3 h further. The solid is collected by filtration, washed with 200 mL of water and dried at 45° C. in a vacuum oven for 36 h to obtain 15.6 g of crystalline (E)-(3R,5S)-(+)-7-[3-(4-fluoro-phenyl)-1-isopropyl-1H-indol-2-yl]-3,5-dihydroxy-hept-6-enoic acid calcium salt as characterized by mp and powder X-ray diagram.

What is claimed is:

1. A calcium salt of the formula 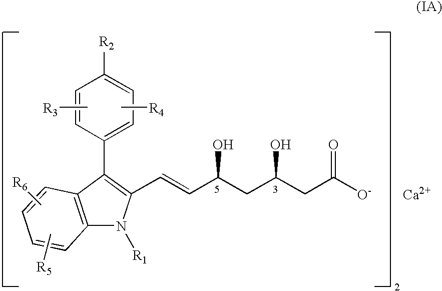
wherein R₁ is alkyl, cycloalkyl or aralkyl; R₂, R₃ and R₄ are independently hydrogen, halogen or alkyl; R₅ and R₆ are independently hydrogen, halogen, alkyl, cycloalkyl, aralkyl, alkoxy or aralkoxy; and the hydroxyl group at the 3-position is in the R-configuration and at the 5-position in the S-configuration; or an enantiomer thereof; or a hydrate thereof; obtainable by a process comprising:
(1) hydrolyzing a compound of the formula 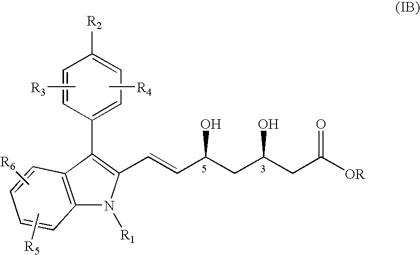
wherein R₁, R₂, R₃, R₄, R₅ and R₆ have meanings as defined for formula IA; R represents lower alkyl; and the hydroxyl group at the 3-position is in the R-configuration and at the 5-position in the S-configuration; or an enantiomer thereof; in the presence of an aqueous base to afford an alkali metal salt of the formula 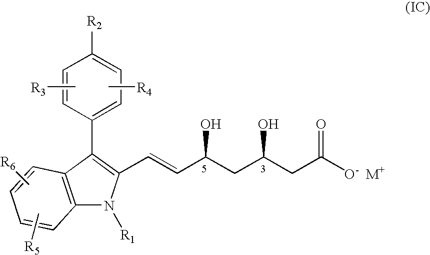
wherein M represents sodium, lithium or potassium; and
(2) treating the alkali metal salt of formula IC with a calcium compound to afford the calcium salt of formula IA.

2. A calcium salt according to claim 1, obtainable by a process wherein the aqueous base in step (1) is sodium hydroxide and M in formula IC represents sodium and wherein the calcium compound in step (2) is calcium chloride.

3. A calcium salt according to claim 1, wherein R₁ is isopropyl, R₂ is fluorine, and R₃, R₄, R₅ and R₆ are hydrogen.

4. A calcium salt of the formula 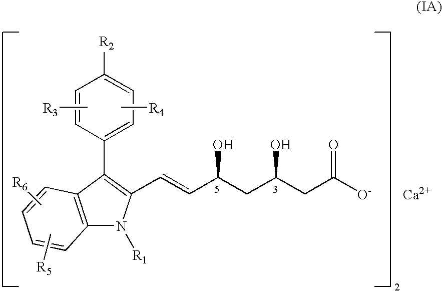
wherein R₁ is alkyl, cycloalkyl or aralkyl; R₂, R₃ and R₄ are independently hydrogen, halogen or alkyl; R₅ and R₆ are independently hydrogen, halogen, alkyl, cycloalkyl, aralkyl, alkoxy or aralkoxy; and the hydroxyl group at the 3-position is in the R-configuration and at the 5-position in the S-configuration; or an enantiomer thereof; or a hydrate thereof; obtainable by treating an alkali metal salt of the formula 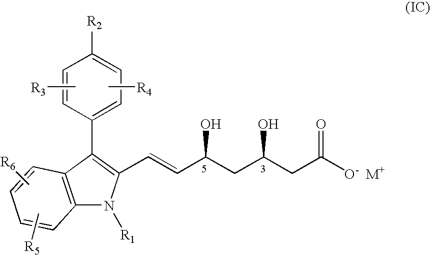
wherein R₁, R₂, R₃, R₄, R₅ and R₅ have meanings as defined for formula IA; M represents sodium, lithium or potassium; and the hydroxyl group at the 3-position is in the R-configuration and at the 5-position in the S-configuration; or an enantiomer thereof; or a hydrate thereof; with a calcium compound to afford the calcium salt of formula IA.

5. A calcium salt according to claim 4, obtainable by a process wherein M in formula IC represents sodium and the calcium compound is calcium chloride.

6. A calcium salt according to claim 4, wherein R₁ is isopropyl, R₂ is fluorine, and R₃, R₄, R₅ and R₅ are hydrogen.

7. A crystalline calcium salt of the formula 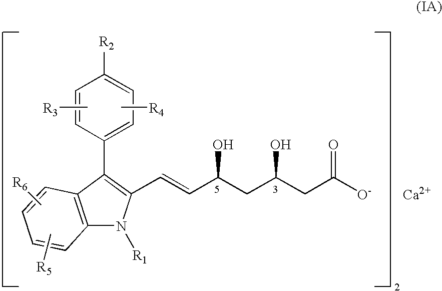
wherein R₁ is isopropyl; R₂ is fluorine; R₃, R₄, R₅ and R₆ are hydrogen; and the hydroxyl group at the 3-position is in the R-configuration and at the 5-position in the S-configuration; or an enantiomer thereof; or a hydrate thereof.

8. A crystalline calcium salt according to claim 7, which has a powder X-ray diffraction pattern with maxima at 2θ values of 5.3, 11.8, 13.9, 17.5, 19.1, 22.0 and 23.1 and which has a melting point of about 220° C.

9. A method for the preparation of a crystalline calcium salt according to claim 7, which method comprises:
(1) hydrolyzing a compound of the formula 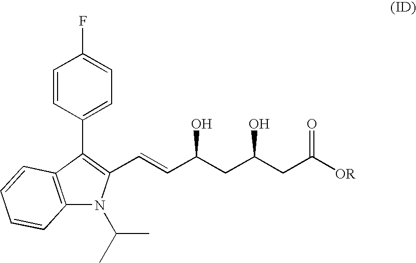
wherein R represents lower alkyl; and the hydroxyl group at the 3-position is in the R-configuration and at the 5-position in the S-configuration; or an enantiomer thereof; in the presence of an aqueous base to afford an alkali metal salt of the formula 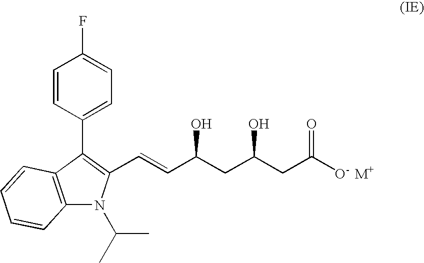
wherein M represents sodium, lithium or potassium; and
(2) treating the alkali metal salt of formula IE with a calcium compound to afford the crystalline calcium salt according to claim 7.

10. The method according to claim 9, wherein the aqueous base in step (1) is sodium hydroxide and M in formula IE represents sodium and wherein the calcium compound in step (2) is calcium chloride.

11. A method for the preparation of a crystalline calcium salt according to claim 7, which method comprises treating an alkali metal salt of the formula 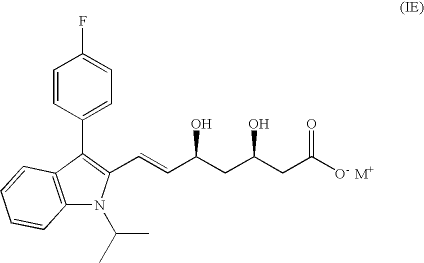
wherein M represents sodium, lithium or potassium; and the hydroxyl group at the 3-position is in the R-configuration and at the 5-position in the S-configuration; or an enantiomer thereof; or a hydrate thereof; with a calcium compound to afford the crystalline calcium salt according to claim 7.

12. The method according to claim 11, wherein M in formula IE represents sodium and the calcium compound is calcium chloride.

13. A pharmaceutical composition comprising a therapeutically effective amount of a calcium salt according to claim 7 in combination with one or more pharmaceutically acceptable carriers.

14. A method for the prevention and/or treatment of hypercholesterolemia, hyperlipoproteinemia, dyslipidemia and atherosclerosis, which method comprises administering to a mammal in need thereof a therapeutically effective amount of a calcium salt according to claim 7.
